(19) United States
(12) Patent Application Publication
(10) Pub. No.: US 2006/0031999 A1
(43) Pub. Date: Feb. 16, 2006

(54) Process for dyeing keratin fibers using a dye composition comprising at least one hydrophobic dye

(76) Inventors: Maxime De Boni, Paris (FR); Sylvain Kravtchenko, Asnieres (FR); Alain Lagrange, Coupvray (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/204,238

(22) Filed: Aug. 16, 2005

Related U.S. Application Data
(60) Provisional application No. 60/645,751, filed on Jan. 24, 2005.

(30) Foreign Application Priority Data
Aug. 16, 2004 (FR) 04 51856

(51) Int. Cl. A61K 8/00

(52) U.S. Cl. 8/405

(57) ABSTRACT

The present disclosure relates to a process for dyeing keratin fibers comprising applying to the keratin fibers a composition comprising at least one hydrophobic direct dye with a logP of greater than 2, in an aqueous-alcoholic dyeing medium comprising less than or equal to 60% water, wherein the dye composition is in contact with the keratin fibers and is exposed to a temperature greater than or equal to 60° C.

This application claims benefit of U.S. Provisional Application No. 60/645,751, filed Jan. 24, 2005, the contents of which are incorporated herein by reference. This application also claims benefit of priority under 35 U.S.C. § 119 to French Patent Application No. 04 51856, filed Aug. 16, 2004, the contents of which are also incorporated herein by reference.

The present disclosure relates to a process for dyeing keratin fibers using a dye composition comprising, in a suitable medium, at least one hydrophobic dye.

Many people have sought for a long time to modify the color of their hair, and for example, to mask their grey hair. To do this, it is known practice to permanently dye keratin fibers by oxidation dyeing. This dyeing technique can consist in applying to the keratin fibers a composition containing dye precursors such as oxidation bases and couplers. These precursors, under the action of at least one oxidizing agent, can form at least one colored species in the hair.

The variety of molecules used as oxidation bases and couplers can allow a rich palette of colors to be obtained. The colorations resulting therefrom can be permanent, strong and resistant to external agents, such as light, bad weather, washing, perspiration and rubbing.

However, the coloration may be performed using oxidizing products such as aqueous hydrogen peroxide solution in basic medium. These oxidizing agents can attack the keratin of the hair, and the cosmetic and mechanical properties of which may become substantially degraded in the case of repeated coloration, which may result in difficulty in styling or shaping.

Moreover, it is known practice to dye keratin fibers, such as human hair, with dye compositions containing direct dyes. The standard dyes that may be used are, for instance, dyes of the nitrobenzene, anthraquinone, nitropyridine, azo, xanthene, acridine, azine or triarylmethane type or natural dyes. These dyes may be nonionic, anionic, cationic or amphoteric. These dyes are colored and coloring molecules with affinity for keratin fibers.

Direct dyes may be generally used in a medium that may comprise predominantly of water, optionally with a minor amount of solvent such as alcohols. Such compositions are described, for example, in European Patent Nos. EP 1 366 752 and EP 1 369 105. It is-also known practice, in order to improve the solubility in water, of using dispersed cationic dyes or solvents by addition of an anionic surfactant, as in shown in U.S. Pat. No. 5,593,459.

These compositions containing at least one direct dye are applied to the keratin fibers and left for a period of time that is necessary to obtain the desired coloration, and are then rinsed out. The colorations resulting therefrom are colorations that may often be chromatic, but that may be temporary or semi-permanent since the nature of the interactions that bind the direct dyes to the keratin fiber, and their desorption from the surface and/or the core of the fiber, are responsible for their low dyeing power and their poor wash-fastness or perspiration-fastness.

Thus, there is still a need to develop novel direct dye compositions that mayobtain varied shades, for example pastel shades, which may show good fastness, for instance with respect to external agents such as the light, shampoo and sweat. For example, there is a need to develop dye compositions for obtaining colorations that can show fastness close to that of oxidation dyeing, without the drawbacks associated with the presence of an oxidizing agent.

The present disclosure relates to a process for dyeing keratin fibers comprising applying to the keratin fibers a composition comprising at least one hydrophobic direct dye with a logP of greater than 2, in an aqueous-alcoholic dyeing medium comprising less than or equal: to 60% water, wherein the composition is in contact with the keratin fibers and is exposed to a temperature greater than or equal to 60° C.

The process of the present disclosure can, in some embodiments, allow shades ranging from pastel to strong colorations to be obtained. Furthermore, the coloration obtained can, in some embodiments, make it possible to achieve or even exceed the color-fastness of oxidation dyeing. Thus, the coloration obtained can, for example, be highly resistant to external agents, such as repeated washing.

As used herein, the logP value is understood to mean the partition coefficient of the dye between octanol and water. The logP value may be calculated according to the method described in the article by Meylan and Howard “Atom/Fragment contribution method for estimating octanol-water partition coefficient”, J. Pharm. Sci., 84: 83-92, 1995. This value may also be calculated using a range of software available on the market that determines the logP value as a function of the structure of a molecule. For example, non-limiting mention may be made of the Epiwin software from the United States Environmental Protection Agency.

The at least one direct dye that can be used in the composition of the present disclosure are chosen from hydrophobic dyes known in the art, which have a logP value of greater than 2. Non-limiting examples that may be mentioned include:

| Dye | Chemical structure | LogP |
|---|---|---|
| Disperse Red 13 | 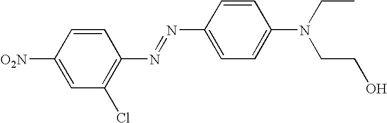 | 5.22 |
| Disperse Green 9 | 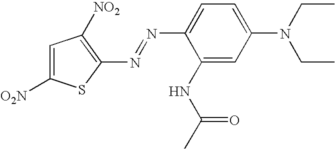 | 4.23 |
| Solvent Black 3 | 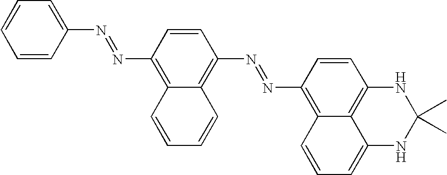 | 7.50 |
| Disperse Blue 148 | 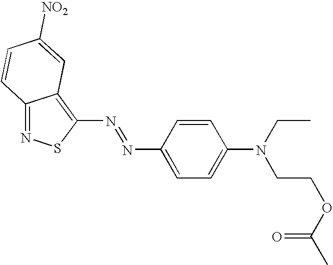 | 4.81 |
| Disperse Violet 63 | 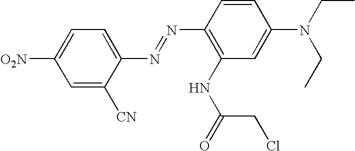 | 5.30 |
| Disperse Blue 60 | 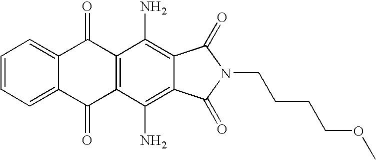 | 3.38 |
| Disperse Blue 14 | 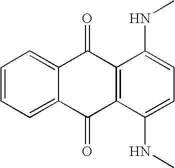 | 4.25 |
| Solvent Orange 15 | 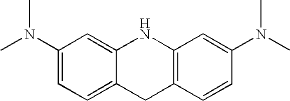 | 3.90 |
| Solvent Orange 7 | 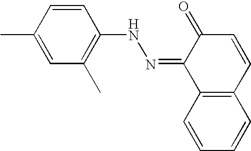 | 4.40 |
| Solvent Blue 14 | 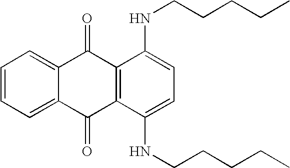 | 8.18 |
| Disperse Yellow 82 | 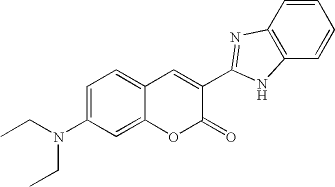 | 3.68 |

According to one embodiment, for example, the logP of the at least one dye used according to the present disclosure is greater than or equal to 4.

The at least one direct dye with a logP of greater than 2 can be present in the composition in an amount ranging from 0.001% to 5% by weight, relative to the total weight of the ready-to-use composition.

Among the alcohols that may be used in the aqueous-alcoholic dyeing medium, non-limiting mention may be made, for example, of C₁-C₆ lower alkanols, and polyols and polyol ethers comprising a free —OH functional group. Further non-limiting examples that may be mentioned include 2-butoxyethanol, propylene glycol, propylene glycol monomethyl ether, diethylene glycol monomethyl ether and monoethyl ether, ethylene glycol monoethyl ether, ethylene glycol monobutyl ether, neopentyl glycol, isoprene glycol, and also aromatic alcohols, for instance benzyl alcohol or phenoxyethanol, phenylethyl alcohol, ethanol, isopropanol, n-propanol, butanol, n-pentanol, 1,2-propanediol, 1,3-propanediol, 1-methoxy-2-propanol, 1-ethoxy-2-propanediol, 1,3- and 1,4-butanediol, and 1,2-hexanediol, and mixtures thereof.

The composition can comprise, for example, water in an amount ranging from 0.1% to 60% by weight, such as from 20% to 60% by weight, and for instance from 40% to 55% by weight, relative to the total weight of the composition.

According to one embodiment of the present disclosure, the amount of alcohol can be greater than or equal to 5%, for example ranging from 10% to 75%. According to another embodiment of the present disclosure, the alcohol is present in an amount ranging from 30% to 60% by weight. The percentages are weight percentages relative to the total weight of the composition used in the process of the present disclosure, unless otherwise indicated.

The composition used in the process of the present disclosure may also comprise at least one additional direct dye other than the at least one hydrophobic direct dye described above. The at least one additional direct dye can be chosen from, for example, neutral, acidic and/or cationic nitrobenzene direct dyes; neutral, acidic and/or cationic azo direct dyes; neutral, acidic and/or cationic quinone, and for instance anthraquinone direct dyes; azine direct dyes; triarylmethane direct dyes; indoamine direct dyes; and natural direct dyes.

Among the benzene-based direct dyes that can be used, mention may be made, in a non-limiting manner, of the following compounds:
1,4-diamino-2-nitrobenzene,
1-amino-2-nitro-4-(β-hydroxyethylamino)benzene,
1-amino-2-nitro-4-bis(β-hydroxyethyl)aminobenzene,
1,4-bis(β-hydroxyethylamino)-2-nitrobenzene,
1-β-hydroxyethylamino-2-nitro-4-bis(β-hydroxyethylamino)benzene,
1-β-hydroxyethylamino-2-nitro-4-aminobenzene,
1-β-hydroxyethylamino-2-nitro-4-(ethyl)(β-hydroxyethyl)aminobenzene,
1-amino-3-methyl-4-β-hydroxyethylamino-6-nitrobenzene,
1-amino-2-nitro-4-β-hydroxyethylamino-5-chlorobenzene,
1,2-diamino-4-nitrobenzene,
1-amino-2-β-hydroxyethylamino-5-nitrobenzene,
1,2-bis(β-hydroxyethylamino)-4-nitrobenzene,
1-amino-2-[tris(hydroxymethyl)methylamino]-5-nitrobenzene,
1-hydroxy-2-amino-5-nitrobenzene,
1-hydroxy-2-amino-4-nitrobenzene,
1-hydroxy-3-nitro-4-aminobenzene,
1-hydroxy-2-amino-4,6-dinitrobenzene,
1-β-hydroxyethyloxy-2-β-hydroxyethylamino-5-nitrobenzene,
1-methoxy-2-β-hydroxyethylamino-5-nitrobenzene,
1-β-hydroxyethyloxy-3-methylamino-4-nitrobenzene,
1-β,γ-dihydroxypropyloxy-3-methylamino-4-nitrobenzene,
1-β-hydroxyethylamino-3-methyl-2-nitrobenzene,
1-β-aminoethylamino-5-methoxy-2-nitrobenzene,
1-hydroxy-2-chloro-6-ethylamino-4-nitrobenzene,
1-hydroxy-2-chloro-6-amino-4-nitrobenzene,
1-hydroxy-6-[bis(β-hydroxyethyl)amino]-3-nitrobenzene,
1-β-hydroxyethylamino-2-nitrobenzene, and
1-hydroxy-4-β-hydroxyethylamino-3-nitrobenzene.

Among the azo direct dyes that may be used, non-limiting mention may be made of the cationic azo dyes described in International Patent Application Nos. WO 95/15144 and WO 95/01772, and European Patent Application No. EP 714 954, the contents of which are incorporated herein by reference.

Further among these compounds, non-limiting mention may be made of the following dyes:
1,3-dimethyl-2-[[4-(dimethylamino)phenyl]azo]-1H-imidazolium chloride,
1,3-dimethyl-2-[(4-aminophenyl)azo]-1H-imidazolium chloride, and
1-methyl-4-[(methylphenylhydrazono)methyl]pyridinium methyl sulfate.

Among the azo direct dyes that may also be used, non-limiting mention may be made of the following dyes described in the Color Index International 3rd edition:
Acid Yellow 9,
Acid Black 1,
Basic Red 22,
Basic Red 76,
Basic Yellow 57,
Basic Brown 16,
Acid Yellow 36,
Acid Orange 7,
Acid Red 33,
Acid Red 35,
Basic Brown 17,
Acid Yellow 23, and
Acid Orange 24.

Mention may also be made of, in a non-limiting manner, 1-(4′-aminodiphenylazo)-2-methyl-4-[bis(β-hydroxyethyl)amino]benzene and 4-hydroxy-3-(2-methoxyphenylazo)-1-naphthalenesulfonic acid.

Among the quinone direct dyes that may be used, non-limiting mention may be made of the following dyes:
Acid Violet 43,
Acid Blue 62,
Basic Blue 22,
Basic Blue 99,
   and also the following compounds:
1-N-methylmorpholiniumpropylamino-4-hydroxyanthraquinone,
1-aminopropylamino-4-methylaminoanthraquinone,
1-aminopropylaminoanthraquinone,
5-β-hydroxyethyl-1,4-diaminoanthraquinone,
2-aminoethylaminoanthraquinone, and
1,4-bis(β,γ-dihydroxypropylamino)anthraquinone.

Among the azine dyes that may be used, non-limiting mention may be made of the following compounds:
Basic Blue 17 and
Basic Red 2.

Among the triarylmethane dyes that may be used, non-limiting mention may be made of the following compounds:
Basic Green 1,
Acid Blue 9,
Basic Violet 3,
Basic Violet 14,
Basic Blue 7,
Acid Violet 49,
Basic Blue 26, and
Acid Blue 7.

Among the natural direct dyes that may be used according to the present disclosure, non-limiting mention may be made of lawsone, juglone, alizarin, purpurin, carminic acid, kermesic acid and spinulosin. Extracts or decoctions comprising these natural dyes may also be used, such as henna-based poultices or extracts.

When the composition according to the present disclosure comprises at least one additional direct dyes other than the at least one direct dye with a logP of greater than or equal to 2, then the composition may comprise less than or equal to 20% of the at least one additional direct dye. According to one embodiment, the composition of the present disclosure can comprise a total amount of the at least one additional direct dye ranging from 0.001% to 10% by weight, relative to the total weight of the composition.

The composition may also comprise at least one oxidation base and/or at least one coupler conventionally used for oxidation dyeing.

Non-limiting examples that may be mentioned include para-phenylenediamines, bis(phenyl)alkylenediamines, para-aminophenols, ortho-aminophenols and heterocyclic bases, and the addition salts thereof.

The couplers may be chosen from, by way of non-limiting example, for instance, meta-phenylenediamine couplers, meta-aminophenol couplers, meta-diphenol couplers, naphthalene-based couplers and heterocyclic couplers, and the addition salts thereof.

When they are present, the at least one oxidation bases and/or coupler can each be present in an amount ranging from 0.001% to 10% by weight, such as from 0.005% to 6% by weight, relative to the total weight of the dye composition.

The composition may also comprise at least one adjuvant conventionally used in hair dye compositions, such as anionic, cationic, nonionic, amphoteric and/or zwitterionic surfactants; anionic, cationic, nonionic, amphoteric and/or zwitterionic polymers; mineral and/or organic thickeners, such as anionic, cationic, nonionic and/or amphoteric polymeric associative thickeners; antioxidants; penetrating agents; sequestering agents; fragrances; buffers; dispersants; conditioners, for instance volatile and/or non-volatile, modified and/or unmodified silicones; film-forming agents; ceramides; preserving agents; and opacifiers.

The at least one adjuvant can be present in an amount for each adjuvant ranging from 0.01% to 20% by weight, relative to the weight of the composition.

For the dyeing of human keratin fibers, the dyeing medium is a cosmetic medium.

Needless to say, a person skilled in the art will take care to select this or these optional additional compound(s) such that the beneficial properties intrinsically associated with the dye composition in accordance with the present disclosure are not, or are not substantially, adversely affected by the envisaged addition(s).

The pH of the dye composition in accordance with the present disclosure can range from 2 to 12, such as from 2 to 7. It may be adjusted to the desired value by means of acidifying or basifying agents usually used for dyeing keratin fibers, or alternatively using standard buffer systems.

Among the acidifying agents that may be used, for example, non-limiting mention may be made of mineral or organic acids, for instance hydrochloric acid, orthophosphoric acid, sulfuric acid, carboxylic acids, for instance acetic acid, tartaric acid, citric acid or lactic acid, and sulfonic acids.

Among the basifying agents that may be used, for example, non-limiting mention may be made of aqueous ammonia, alkaline carbonates, alkanolamines such as monoethanolamine, diethanolamine and triethanolamine and derivatives thereof, sodium hydroxide, potassium hydroxide and the compounds of formula (III) below:

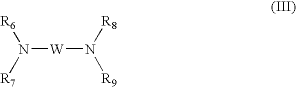

wherein W is a propylene residue optionally substituted with a hydroxyl group or a C₁-C₄ alkyl radical; R₆, R₇, R₈ and R₉, which may be identical or different, are chosen from hydrogen atoms, C₁-C₄ alkyl and C₁-C₄ hydroxyalkyl radicals.

In the process of the present disclosure, the period of leave-in time can range from 1 to 60 minutes, such as from 10 to 60 minutes. After the period of leave-in time, the keratin fibers are rinsed to reveal dyed fibers.

The temperature of greater than or equal to 60° C. may be obtained by various means, such as a hairdryer, a hood or curling or straightening tongs. According to one embodiment of the present disclosure, the temperature of the composition on the keratin fibers can range from 60° C. to 250° C., such as from 60° C. to 100° C.

The dyeing composition may also optionally comprise at least one oxidizing agent either for lightening the fibers, or for when an oxidation base and/or a coupler are/is used in the composition. The oxidizing agents conventionally used for the oxidation dyeing of keratin fibers are, for example, hydrogen peroxide, urea peroxide, alkali metal bromates, persalts such as perborates and persulfates, peracids, and oxidase enzymes, among which non-limiting mention may be made of peroxidases, 2-electron oxidoreductases such as uricases, and 4-electron oxygenases, for instance laccases. In one embodiment of the present disclosure, hydrogen peroxide is used as an oxidizing agent.

The at least one oxidizing agent may be added to the composition of the present disclosure just at the time of use, or it may be used starting with at least one oxidizing composition comprising it, which is applied simultaneously with or sequentially to the composition of the present disclosure. The at least one oxidizing composition may also comprise at least one adjuvant conventionally used in hair dye compositions and as defined above.

The pH of the at least one oxidizing composition comprising the oxidizing agent is such that, after mixing with the at least one dye composition, the pH of the resulting composition applied to the keratin fibers ranges from 3 to 12, such as from 5 to 11. It may be adjusted to the desired value by means of acidifying or basifying agents usually used in the dyeing of keratin fibers and as defined above.

The composition that is finally applied to the keratin fibers can be in various forms, such as in the form of liquids, creams or gels, or in any other form that is suitable for dyeing keratin fibers, for instance human hair.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The examples that follow serve to illustrate the present disclosure without, however, being limiting in nature.

### EXAMPLES

### Example 1

A water/ethanol (1:1) solution comprising 0.3% Disperse Red 13 was applied to locks of natural grey hair comprising 90% white hairs, and to locks of permanent-waved grey hair comprising 90% white hairs.

After application and contact with the keratin fibers for a period of 30 minutes, with the temperature at 60° C., the locks were rinsed and dried. The locks thus treated were dyed red.

What is claimed is:

1. A process for dyeing keratin fibers comprising applying to the keratin fibers a dye composition comprising at least one hydrophobic direct dye with a logP of greater than 2, in an aqueous-alcoholic dyeing medium comprising less than or equal to 60% water, wherein the dye composition is in contact with the keratin fibers and is exposed to a temperature of greater than or equal to 60° C.

2. The process according to claim 1, wherein the at least one hydrophobic direct dye has a logP of greater than or equal to 4.

3. The process according to claim 1, wherein the at least one alcohol present in the composition is chosen from C₁-C₆ lower alkanols, polyols, polyol ethers, and mixtures thereof.

4. The process according to claim 3, wherein the at least one alcohol is chosen from 2-butoxyethanol, propylene glycol, propylene glycol monomethyl ether, diethylene glycol monoethyl ether, diethylene glycol monomethyl ether, ethylene glycol monoethyl ether, ethylene glycol monobutyl ether, neopentyl glycol, isoprene glycol, aromatic alcohols, phenylethyl alcohol, ethanol, isopropanol, n-propanol, butanol, n-pentanol, 1,2-propanediol, 1,3-propanediol, 1-methoxy-2-propanol, 1-ethoxy-2-propanediol, 1,3-butanediol, 1,4-butanediol, 1,2-hexanediol, and mixtures thereof.

5. The process according to claim 1, wherein the aqueous-alcoholic dyeing medium is present in an amount ranging from 0.1 % to 60% by weight, relative to the total weight of the dye composition.

6. The process according to claim 1, wherein the water is present in the dye composition in an amount ranging from 20% to 60% by weight, relative to the weight of the dye composition.

7. The process according to claim 6, wherein the water is present in an amount ranging from 40% to 55% by weight, relative to the weight of the dye composition.

8. The process according to claim 1, wherein the alcohol is present in the aqueous-alcoholic dyeing medium in an amount greater than or equal to 5% by weight, relative to the weight of the aqueous-alcoholic dyeing medium.

9. The process according to claim 8, wherein the alcohol is present in an amount ranging from 10% to 75% by weight, relative to the weight of the aqueous-alcoholic dyeing medium.

10. The process according to claim 9, wherein the alcohol is present in an amount ranging from 30% to 60% by weight, relative to the weight of the aqueous-alcoholic dyeing medium.

11. The process according to claim 1, wherein the at least one direct dye with a logP of greater than 2 is present in the dye composition in an amount ranging from 0.001% to 5% by weight, relative to the total weight of the dye composition.

12. The process according to claim 1, wherein the composition further comprises at least one additional direct dye chosen from neutral, acidic and/or cationic nitrobenzene direct dyes; neutral, acidic and/or cationic azo direct dyes; neutral, acidic and/or cationic quinone direct dyes; azine direct dyes; triarylmethane direct dyes; indoamine direct dyes; and natural direct dyes.

13. The process according to claim 1, wherein the composition further comprises at least one oxidation base chosen from para-phenylenediamines, bis(phenyl)alkylenediamines, para-aminophenols, ortho-aminophenols, heterocyclic bases, and the addition salts thereof.

14. The process according to claim 1, wherein the composition comprises at least one coupler chosen from meta-phenylenediamines, meta-aminophenols, meta-diphenols, naphthalene-based couplers, heterocyclic couplers, and the addition salts thereof.

15. The process according to claim 13, wherein the at least one oxidation base is present in an amount ranging from 0.001% to 10% by weight, relative to the total weight of the dye composition.

16. The process according to claim 14, wherein each coupler is present in an amount ranging from 0.001% to 10% by weight, relative to the total weight of the dye composition.

17. The process according to claim 1, wherein the dye composition further comprises at least one adjuvant chosen from anionic, cationic, nonionic, amphoteric and/or zwitterionic surfactants; anionic, cationic, nonionic, amphoteric and/or zwitterionic polymers; mineral and/or organic thickeners; anionic, cationic, nonionic and/or amphoteric polymeric associative thickeners; antioxidants; penetrating agents; sequestering agents; fragrances; buffers; dispersants; conditioning agents; film-forming agents; ceramides; preserving agents; and opacifiers.

18. The process according to claim 17, wherein each adjuvant is present in an amount ranging from 0.01% to 20% by weight, relative to the weight of the dye composition.

19. The process according to claim 1, wherein the pH of the dye composition ranges from 2 to 12.

20. The process according to claim 1, wherein the dye composition is in contact with the keratin fibers for a period of time ranging from 1 minute to 60 minutes.

21. The process according to claim 1, wherein the temperature of the dye composition ranges from 60° C. to 250° C.

22. The process according to claim 21, wherein the temperature of the dye composition ranges from 60° C. to 100° C.

23. The process according to claim 1, wherein the dye composition is applied to the keratin fibers while being exposed to a temperature of greater than or equal to 60° C.

24. The process according to claim 1, wherein the dye composition is exposed to a temperature of greater than or equal to 60° C. following contact with the keratin fibers.

25. The process according to claim 1, wherein the dye composition is exposed to a temperature ranging from 60° C. to 250° C.

26. The process according to claim 25, wherein the dye composition is exposed to a temperature ranging from 60° C. to 100° C.
